(19) United States
(12) Patent Application Publication
(10) Pub. No.: US 2021/0298439 A1
(43) Pub. Date: Sep. 30, 2021

(54) SANITIZER SYSTEMS WITH REUSABLE WIPE STORAGE

(71) Applicant: THE FINAL CO. LLC, Santa Fe, NM (US)

(72) Inventors: Emma R. Cohen, Santa Fe, NM (US); Matthew D. Hantke, Ventura, CA (US); William Travis Smith, Fort Collins, CO (US)

(21) Appl. No.: 17/209,421

(22) Filed: Mar. 23, 2021

Related U.S. Application Data
(60) Provisional application No. 63/002,239, filed on Mar. 30, 2020.

(51) Int. Cl. A45C 11/00; A45C 13/02; A47K 10/42; B05B 11/00; A61L 2/26; A61L 2/22

(52) U.S. Cl. CPC A45C 11/008; A45C 13/02; A47K 10/426; B05B 11/0037; A61L 2202/15; A61L 2/22; A45C 2011/007; A61L 2202/17; A61L 2/26

(57) ABSTRACT

Sanitizer systems with reusable wipe storage are disclosed. In accordance with an example, a sanitizer system includes a sanitizer dispenser and a pair of wipe storage trays. The sanitizer dispenser has a sanitizer container adapted to contain sanitizer and a mechanical sprayer coupled to the sanitizer container and adapted to dispense the sanitizer from the sanitizer container. The pair of wipe storage trays are slidably coupled to opposing sides of the sanitizer dispenser and each defining define a wipe storage compartment to store either clean wipes or soiled wipes.

### CROSS-REFERENCE TO RELATED APPLICATION

Priority is claimed to U.S. Provisional Patent Application No. 63/002,239, filed Mar. 30, 2020, the entire contents of which are hereby incorporated by reference herein.

### FIELD OF THE DISCLOSURE

The present patent relates generally to sanitizer systems and, in particular, to sanitizer systems with reusable wipe storage.

### BACKGROUND

Disposable antibacterial wipes are often used to sanitize objects, reduce bacteria, clean people's hands, or for general cleaning. Due to the disposable nature of disposable wipes, each wipe may only be used once before being disposed of. Thus, the use of disposable wipes adds to landfill waste and requires significant amounts of trees and water to constantly replace the disposed of wipes which are made of paper products.

### SUMMARY

Example sanitizer systems include reusable wipes that address the problems of known disposable wipes. Moreover, the example sanitizer systems are cost effective by not requiring the reusable wipes to be frequently replaced. The sanitizer systems include a sanitizer dispenser to dispense sanitizer and a pair of reusable wipe storage compartments. The sanitizer dispenser may include a spray bottle or associated components to store and dispense sanitizer and the reusable wipe storage compartments may be used to store either of clean wipes or soiled wipes.

The sanitizer dispenser may include a mechanical sprayer that, when actuated, dispenses sanitizer. One of the wipe storage compartments may be designated for clean wipes and the other of the wipe storage compartments may be designed for soiled wipes. In an example, the reusable wipe storage compartments are disposed on either side of the sanitizer dispenser. Thus, the sanitizer dispenser may be centrally located.

The reusable wipe storage compartments may be defined by wipe storage trays or by the sanitizer dispenser itself. When the sanitizer system includes the wipe storage trays, the wipe storage trays may be slidably coupled to the sanitizer dispenser such that when the trays are slid relative to a longitudinal axis of the sanitizer dispenser, the corresponding tray is uncovered allowing access to the reusable wipe storage compartment. When the reusable wipe storage compartments are defined by the sanitizer dispenser, doors may selectively cover the reusable wipe storage compartments allowing or preventing access therein.

In accordance with a first example, a sanitizer system includes a sanitizer dispenser and a pair of wipe storage trays. The sanitizer dispenser has a sanitizer container adapted to contain sanitizer and a mechanical sprayer coupled to the sanitizer container and adapted to dispense the sanitizer from the sanitizer container. The pair of wipe storage trays are slidably coupled to opposing sides of the sanitizer dispenser and each define a wipe storage compartment to store either clean wipes or soiled wipes.

In accordance with a second example, a sanitizer system includes a sanitizer dispenser and a pair of doors. The sanitizer dispenser has a sanitizer container adapted to contain sanitizer and a mechanical sprayer coupled to the sanitizer container and adapted to dispense the sanitizer from the sanitizer container. Opposing sides of the sanitizer container each define a wipe storage compartment adapted to store either clean wipes or soiled wipes. The pair of doors are coupled to the sanitizer dispenser and are adapted to cover one of the wipe storage compartments.

In further accordance with the foregoing first and/or second examples, an apparatus may further include any one or more of the following:

In accordance with an example, the sanitizer system includes a plurality of wipes to be disposed in at least one of the wipe storage trays.

In accordance with another example, each of the wipe storage trays is slidable between a closed position preventing access to the corresponding wipe storage compartment and an open position allowing access to the corresponding wipe storage compartment.

In accordance with another example, in the closed position, a perimeter of each of the wipe storage trays and a perimeter of the sanitizer container substantially align.

In accordance with another example, in the closed position, a seal is formed between at least one of the wipe storage trays and the sanitizer container.

In accordance with another example, each wipe storage tray is slidably coupled to corresponding sides of the sanitizer container by a pair of tongue-and-groove connections.

In accordance with another example, each wipe storage tray includes a base and a rim including a pair of opposing side walls and a pair of opposing end walls.

In accordance with another example, the sanitizer container includes a pair of opposing side walls and a pair of opposing end walls that form a perimeter of the sanitizer container. The side walls and the end walls of the sanitizer container align with the rims of the wipe storage trays when the wipe storage trays are in the closed position.

In accordance with another example, a tongue-and-groove connection is formed between each side wall of the wipe storage trays and a corresponding side wall of the sanitizer container.

In accordance with another example, a stop is formed between each wipe storage tray and the sanitizer container.

In accordance with another example, the stop is formed by a pair of corresponding steps.

In accordance with another example, the sanitizer system is a rectangular prism.

In accordance with another example, the mechanical sprayer is a trigger sprayer.

In accordance with another example, the mechanical sprayer is a fine mist sprayer.

In accordance with another example, each of the doors is movable between a closed position preventing access to the corresponding wipe storage compartment and an open position allowing access to the corresponding wipe storage compartment.

In accordance with another example, each door is coupled to the sanitizer container via a hinge.

In accordance with another example, the sanitizer system includes a pair of latches with each latch being used to secure one of the doors in the closed position.

In accordance with another example, each wipe storage compartment has an opening and each latch includes a tab operatively coupled to the sanitizer dispenser and extending over the opening and adapted to secure the door in the closed position.

In accordance with another example, the doors are coupled to the sanitizer container via a living hinge.

In accordance with another example, the sanitizer container has an I-beam cross-section.

In accordance with another example, the sanitizer container includes a web and flanges that form the wipe storage compartments.

In accordance with another example, the sanitizer dispenser includes a clam-shell case that surrounds the sanitizer container and defines an opening from which the mechanical sprayer extends.

In accordance with another example, the clam-shell case is coupled via a snap-fit connection.

In accordance with another example, one or more of the doors has an access opening.

In accordance with another example, the sanitizer system includes a gasket coupled to the door and defining the access opening.

### BRIEF DESCRIPTION OF THE DRAWINGS

FIG. 1 is an isometric view of a sanitizer system in accordance with a first example of the present disclosure.

FIG. 2 is an isometric view of the sanitizer system of FIG. 1 including a lanyard.

FIG. 3 is a cross-sectional view of the sanitizer system of FIG. 2 in the closed position showing the wipe storage trays and the sanitizer container.

FIG. 4 is a cross-sectional view of the sanitizer system of FIG. 2 with both of the swipe storage trays in the open position.

FIG. 5 is an cross-sectional view of a sanitizer system in accordance with a second example of the present disclosure.

FIG. 6 is an isometric view of a sanitizer system in accordance with a third example of the present disclosure.

FIG. 7 is an isometric view of the door that can be used with the sanitizer system of FIG. 6.

### DETAILED DESCRIPTION

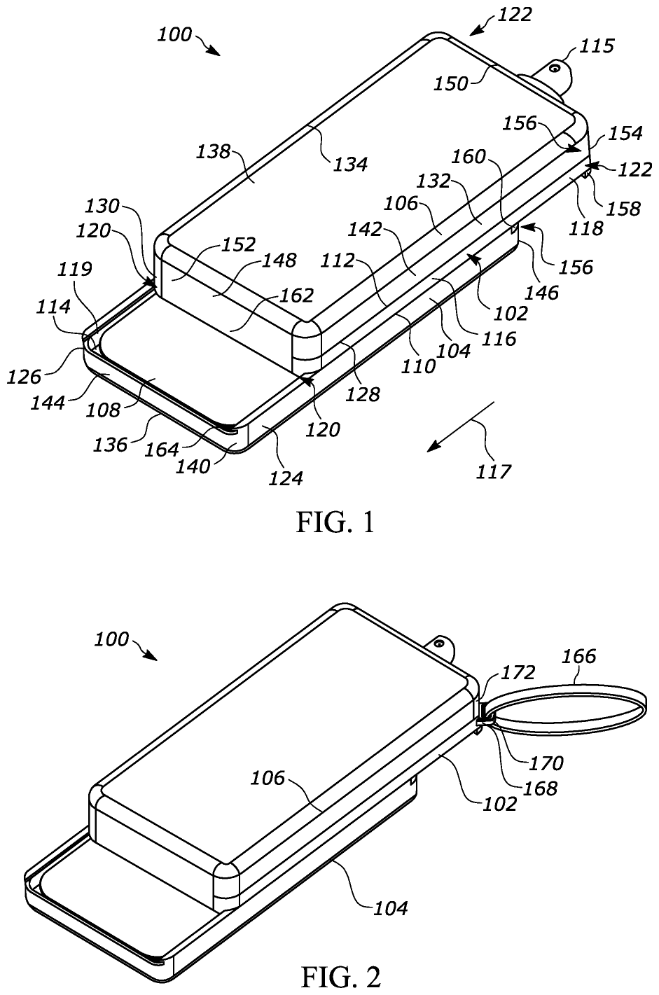

Turning to FIG. 1, a sanitizer system 100 is shown in accordance with a first example of the present disclosure. The sanitizer system 100 may be conveniently sized for travel applications and is used to dispense sanitizer and to store both clean reusable wipes and soiled reusable wipes. To do so, the sanitizer system 100 includes a sanitizer dispenser 102 and a pair of wipe storage trays 104, 106.

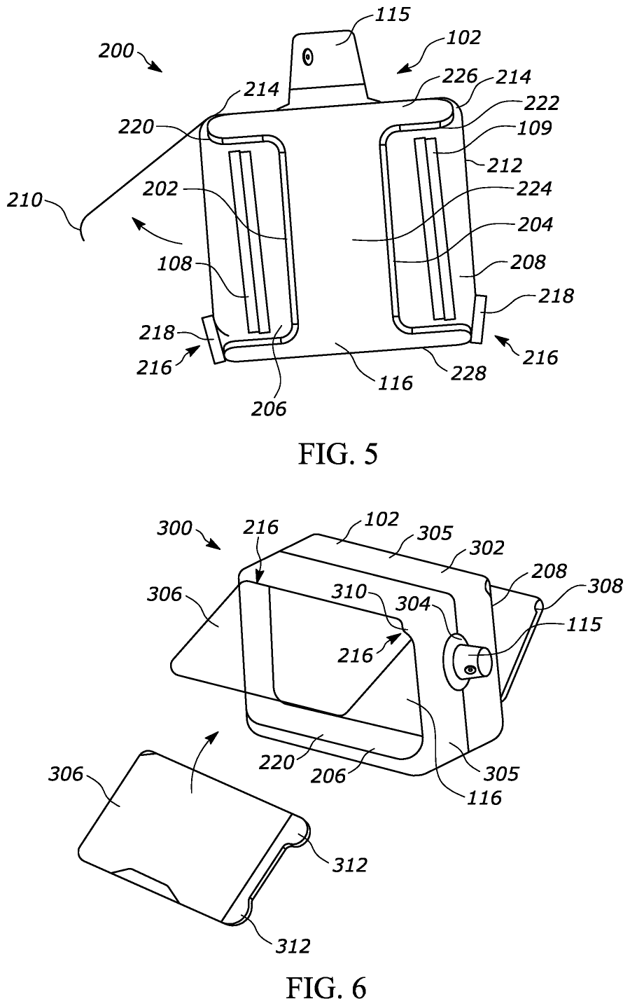

The sanitizer dispenser 102 stores and dispenses sanitizer and the wipe storage trays 104, 106 are adapted to store a clean wipe(s) 108 and/or a soiled wipe(s) 109 (see, FIG. 5). In the example shown, the wipe storage trays 104, 106 are slidably coupled to opposing sides 110, 112 of the sanitizer dispenser 102, thereby allowing or preventing access to a wipe storage compartment 114 (one of which is shown in FIG. 1) defined by the corresponding wipe storage trays 104, 106.

To disinfect a surface using the sanitizer system 100, one of the clean wipes 108 may be removed from the clean wipe storage tray 104, the sanitizer dispenser 102 may be used to spray sanitizer on the surface, and the clean wipe 108 may be used to wipe and sanitize the surface. To spray the sanitizer, the sanitizer dispenser 102 includes a mechanical sprayer 115 which may be formed as a trigger sprayer or a fine mist sprayer.

Once the surface is sanitized using the sanitizer and the wipe 108, the now soiled wipe may be placed in the soiled wipe storage tray 106. To access either of the wipe storage trays 104, 106, the wipe storage trays 104, 106 are movable between an open position (see, the clean wipe storage tray 104) and a closed position (see, the soiled wipe storage tray 106).

A perimeter of the wipe storage tray 104, 106 and a perimeter of a sanitizer container 116 of the sanitizer dispenser 102 substantially align when the wipe storage trays 104, 106 are in the closed position. Thus, the sanitizer system 100 is conveniently sized to be stored in a bag, a pocket, etc. A seal 118 may formed between the soiled wipe storage tray 106 and the sanitizer container 116 when the soiled wipe storage tray 106 and the sanitizer container 116 align. The seal 118 may prevent sanitizer from leaking from the soiled wipe storage tray 106 if, for example, one of the soiled wipes is saturated with sanitizer. A seal may also be formed between the clean wipe storage tray 104 and the sanitizer container 116 or the seal 118 may be omitted.

To uncover an opening 119 of the clean wipe storage tray 104 and allow one of the clean wipes 108 to be removed from the sanitizer system 100, the clean wipe storage tray 104 may be moved in a direction generally indicated by arrow 117 relative to the sanitizer dispenser 102. Similarly, the soiled wipe storage tray 106 may be moved relative to the sanitizer dispenser 102 to uncover an opening of the soiled wipe storage tray 106 and allow the soiled wipe to be deposited within the soiled wipe storage tray 106 for storage or for the soiled wipes 109 to be removed for cleaning. Thus, each of the wipe storage trays 104, 106 is slidable between a closed position preventing access to the corresponding wipe storage compartment 114 and an open position allowing access to the corresponding wipe storage compartment 114. The soiled wipe storage tray 106 may be opened by moving the soiled wipe storage tray 106 in the direction generally indicated by arrow 117 or in a direction generally opposite that of the direction indicated by the arrow 117.

In the example shown, the sanitizer container 116 of the sanitizer dispenser 102 is formed substantially as a rectangular prism having the opposing sides 110, 112 that provide mounting surfaces for the wipe storage trays 104. Each wipe storage tray 104, 106 is slidably coupled to a corresponding side 110, 112 of the sanitizer container 116 by a pair of tongue-and-groove connections 120, 122. The tongue-and-groove connections 120 between the clean wipe storage tray 104 and the sanitizer container 116 are formed between side walls 124, 126 of the clean wipe storage tray 104 and side walls 128, 130 of the sanitizer container 116. Similarly, the tongue-and-groove connections 122 between the soiled wipe storage tray 106 and the sanitizer container 116 are formed between side walls 132, 134 of the soiled wipe storage tray 106 and the side walls 128, 130 of the sanitizer container 116.

Referring to the wipe storage trays 104,106, each of the wipe storage trays 104, 106 includes a base 136, 138 and a rim 140, 142. The rims 140, 142 extend from the corresponding base 136, 138 and are coupled to one of the sides 110, 112 of the sanitizer container 116. Each rim 140, 142 includes the pair of opposing side walls 124, 126, 132, 134 and a pair of opposing end walls 144, 146, 148, 150. The sanitizer container 116 also includes the pair of opposing side walls 128, 130 and a pair of opposing end walls 152, 154 that form a perimeter of the sanitizer container 116. As disclosed above, the side walls 128, 130 and the end walls 152, 154 of the sanitizer container 116 align with the rims 140, 142 of the wipe storage trays 104, 106 when the wipe storage trays 104, 106 are in the closed position.

Referring still to FIG. 1, a stop 156 may be formed between each wipe storage tray 104, 106 and the sanitizer container 116. The stop 156 may be used to align a perimeter of the wipe storage trays 104, 106 and a perimeter of the sanitizer container 116 when the wipe storage trays 104, 106 are in the closed position. In the example shown, the stop 156 between the clean wipe storage tray 104 and the sanitizer container 116 is formed by a pair of corresponding steps 158, 160. The steps 158, 160 are adapted to engage and prevent further movement of the sanitizer container 116 in the direction generally indicated by arrow 117.

The engagement between the steps 158, 160 may also close an opening that may otherwise be present between the clean wipe storage tray 104 and the sanitizer container 116 at the end walls 146, 154. The step 160 of the sanitizer container 116 may be formed as a lip or a protrusion that covers the opening that may extend across a width of the clean wipe storage tray 104. On the opposing end of the sanitizer system 100, the end wall 152 of the sanitizer container 116 includes a protrusion 162 that is adapted to be disposed within a cutout 164 of the clean wipe storage tray 104 formed between the side walls 124, 126 and the end wall 144 of the clean wipe storage tray 104 when the components 116, 104 align.

In practice, the sanitizer dispenser 102 may be filled with sanitizer by unthreading the mechanical sprayer 115 from the sanitizer container 116 and depositing water and a disinfecting tablet or a portion thereof into the sanitizer container 116. The sanitizer container 116 may have an opening large enough to easily receive at least a portion of the disinfecting tablet. The sanitizer container 116 may store any amount of sanitizer such as, for example, 60 milliliters (mL), 100 mL, 150 mL, 160 mL, etc.

Once the disinfecting tablet and the water are added to the sanitizer container 116, the dip tube (not shown) of the mechanical sprayer 115 is repositioned within the sanitizer container 116, the mechanical sprayer 115 is recoupled to the sanitizer container 116, and the sanitizer dispenser 102 may be shaken to dissolve the disinfecting tablet in the water to form the sanitizer. In another example, the sanitizer may be added to the sanitizer container 116 in liquid form such using, for example, a commercially available sanitizer, disinfecting solution, bleach, etc.

To clean the wipes 108, the wipes 108 may be removed from the wipe storage trays 104, 106 and laundered. The wipes 108 may be made of any type of material including, for example, recycled cotton, cotton, synthetic materials, hemp, bamboo fabric, and/or fabrics having anti-microbial features. In an example, the fabrics having the anti-microbial features may include silver. The wipes 108 may be durable, reusable, soft, absorbent, and/or machine washable.

FIG. 2 is an isometric view of the sanitizer system 100 of FIG. 1 including a lanyard 166. In the example shown, the sanitizer dispenser 102 includes an extension 168 that defines an eyelet 170 through which the lanyard 166 is disposed to couple the lanyard 166 to the sanitizer system 100. The eyelet 170 is shown extending from a corner 172 of the sanitizer dispenser 102 and being oblong but the eyelet 170 may be differently positioned and/or have a different shape.

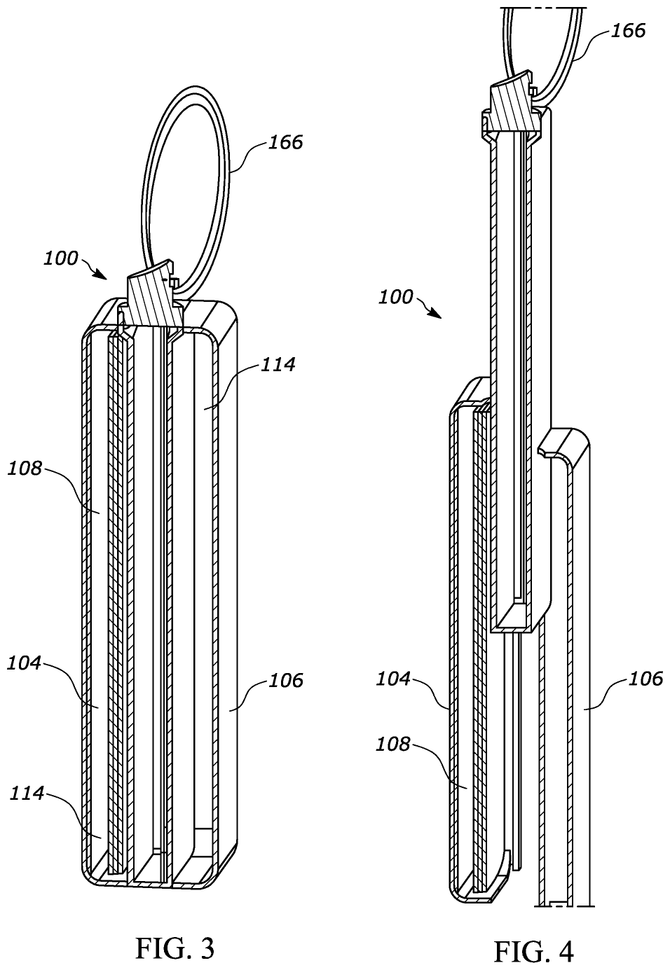

FIG. 3 is a cross-sectional view of the sanitizer system 100 of FIG. 2 in the closed position showing the wipe storage trays 104, 106 and the sanitizer container 116.

FIG. 4 is a cross-sectional view of the sanitizer system of FIG. 2 with both of the swipe storage trays 104, 106 in the open position. As such, the sanitizer container 116 is shown moved away from the wipe storage trays 104, 106 such that the sanitizer container 116 is not in alignment with the wipe storage trays 104, 106.

FIG. 5 is an cross-sectional view of a sanitizer system 200 in accordance with a second example of the present disclosure. Like the sanitizer system 100 of FIG. 1, the sanitizer system 200 of FIG. 5 is used to dispense sanitizer and to store both clean reusable wipes and soiled reusable wipes. To do so and in slight contrast to the sanitizer system 100 of FIG. 1, the sanitizer system 200 of FIG. 5 includes the sanitizer dispenser 102 having opposing surfaces 202, 204 that define wipe storage compartments 206, 208.

As disclosed above, the sanitizer dispenser 102 stores and dispenses sanitizer and the wipe storage compartments 206, 208 are adapted to store the clean wipe(s) 108 and/or the soiled wipe(s) 109. In the example shown, a pair of doors 210, 212 are coupled to the sanitizer dispenser 102 and are adapted to cover the wipe storage compartments 206, 208.

The doors 210, 212 are movable between a closed position preventing access to the corresponding wipe storage compartment 206, 208 and an open position allowing access to the corresponding wipe storage compartment 206, 208. As shown, the clean wipe door 210 is shown partially open to allow one of the clean wipes 108 to be removed.

The doors 210, 212 may be coupled to the sanitizer dispenser 102 in different ways. For example, the doors 210, 212 may be hingably coupled to the sanitizer dispenser 102 or the doors 210, 212 may be removably coupled to the sanitizer dispenser 102. In the example shown, the doors 210, 212 are coupled to the sanitizer dispenser 102 via hinges 214. The hinge 214 may be a living hinge, a pin hinge, or any other type of hinge.

The sanitizer system 200 may also include a pair latches 216 that are used to secure the doors 210, 212 in the closed position. In the example shown, each latch 216 includes a tab 218 that extends over an opening 220, 222 of the corresponding wipe storage compartment 206, 208. The tabs 218 may be flexible such that the doors 210, 212 may move over the tabs 218 and thereafter be secured in the closed position. However, the doors 210, 212 may be secured in different ways. For example, the doors may be secured in the closed position using magnets, a fastener such as a hook-and-loop fastener, etc.

Referring now to the sanitizer container 116 of FIG. 5, in the example shown, the sanitizer container 116 has an I-beam cross-section and includes the surfaces 202, 204 that at least partially define the wipe storage compartments 206, 208. With the I-beam cross-section, the sanitizer container 116 includes a web 224 and flanges 226, 228 that form the wipe storage compartments 206, 208. The sanitizer container 116 may be formed by blow molding. However, other manufacturing techniques may be used instead.

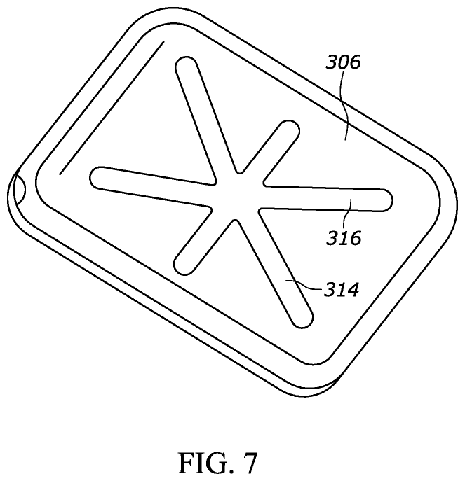

FIG. 6 is an isometric view of a sanitizer system 300 in accordance with a third example of the present disclosure. Like the sanitizer system 100 of FIG. 1 and the sanitizer system 200 of FIG. 5, the sanitizer system 300 of FIG. 6 is used to dispense sanitizer and to store both clean reusable wipes and soiled reusable wipes. To do so and similar to the sanitizer system 200 of FIG. 5, the sanitizer system 300 of FIG. 6 includes the sanitizer dispenser 102 that defines the wipe storage compartments 206, 208. However, in contrast to the sanitizer system 200 of FIG. 5, the sanitizer dispenser 102 of FIG. 6 includes a clam-shell case 302 that surrounds the sanitizer container 116 and defines an opening 304 from which the mechanical sprayer 115 extends. Portions 305 of the clam-shell case 302 may be coupled in any suitable way such as vis fasteners. Some fasteners that may be used form a snap-fit connection.

In the example shown, a pair of doors 306, 308 are removably coupled to the sanitizer dispenser 102 and are adapted to cover the wipe storage compartments 206, 208. The latches 216 may be used to secure the doors 306, 308 to the sanitizer dispenser 102. The latches 216 of FIG. 6 include overlapping tabs 310, 312, where the outer tab 310 is coupled to the sanitizer dispenser 102 and is adapted to extend over the corresponding opening 220, 224 and the inner tab 312 is coupled to the door 306 and is adapted to be positioned underneath the outer tabs 310 to secure the corresponding door 306, 308 in place.

FIG. 7 is an isometric view of the door 306 that can be used with the sanitizer system 300 of FIG. 6. In the example shown, the door 306 includes an access opening 314 that allows the wipes 108 to be removed therethrough. The access opening 314 is shown as being star shaped, however, the access opening 314 may have a different shape. For example, the access opening 314 may be rectangular or circular or have any abstract shape. A gasket 316 may be coupled to the door 306 and define the access opening 314. The gasket 316 may reduce the likelihood that liquid leaks from the corresponding wipe storage compartment 206, 208. While the example of FIG. 7 mentions the door 306, the access opening 314 and/or the gasket 316 may be included with the door 308.

Although the foregoing text discloses a detailed description of example methods, apparatus and/or articles of manufacture, it should be understood that the legal scope of the property right is defined by the words of the claims set forth at the end of this patent. Accordingly, the following detailed description is to be construed as examples only and does not describe every possible example, as describing every possible example would be impractical, if not impossible. Numerous alternative examples could be implemented, using either current technology or technology developed after the filing date of this patent. It is envisioned that such alternative examples would still fall within the scope of the claims.

Further, while several examples have been disclosed herein, any features from any examples may be combined with or replaced by other features from other examples. Moreover, while several examples have been disclosed herein, changes may be made to the disclosed examples without departing from the scope of the claims.

What is claimed is:

1. A sanitizer system comprising:
a sanitizer dispenser having a sanitizer container adapted to contain sanitizer and a mechanical sprayer coupled to the sanitizer container and adapted to dispense the sanitizer from the sanitizer container; and
a pair of wipe storage trays slidably coupled to opposing sides of the sanitizer dispenser and each defining a wipe storage compartment to store either clean wipes or soiled wipes.

2. The sanitizer system of claim 1, further comprising a plurality of wipes to be disposed in at least one of the wipe storage trays.

3. The sanitizer system of claim 1, wherein each of the wipe storage trays is slidable between a closed position preventing access to the corresponding wipe storage compartment and an open position allowing access to the corresponding wipe storage compartment.

4. The sanitizer system of claim 3, wherein in the closed position, a perimeter of each of the wipe storage trays and a perimeter of the sanitizer container substantially align.

5. The sanitizer system of claim 3, wherein in the closed position, a seal is formed between at least one of the wipe storage trays and the sanitizer container.

6. The sanitizer system of claim 1, wherein each wipe storage tray is slidably coupled to corresponding sides of the sanitizer container by a pair of tongue-and-groove connections.

7. The sanitizer system of claim 1, wherein each wipe storage tray includes a base and a rim including a pair of opposing side walls and a pair of opposing end walls.

8. The sanitizer system of claim 7, wherein the sanitizer container includes a pair of opposing side walls and a pair of opposing end walls that form a perimeter of the sanitizer container, the side walls and the end walls of the sanitizer container align with the rims of the wipe storage trays when the wipe storage trays are in the closed position.

9. The sanitizer system of claim 8, wherein a tongue-and-groove connection is formed between each side wall of the wipe storage trays and a corresponding side wall of the sanitizer container.

10. The sanitizer system of claim 1, wherein a stop is formed between each wipe storage tray and the sanitizer container.

11. The sanitizer system of claim 1, wherein the mechanical sprayer is a trigger sprayer and/or a fine mist sprayer.

12. A sanitizer system, comprising:
a sanitizer dispenser having a sanitizer container adapted to contain sanitizer and a mechanical sprayer coupled to the sanitizer container and adapted to dispense the sanitizer from the sanitizer container, wherein opposing sides of the sanitizer container each define a wipe storage compartment adapted to store either clean wipes or soiled wipes; and
a pair of doors coupled to the sanitizer dispenser and adapted to cover one of the wipe storage compartments.

13. The sanitizer system of claim 12, wherein each of the doors is movable between a closed position preventing access to the corresponding wipe storage compartment and an open position allowing access to the corresponding wipe storage compartment.

14. The sanitizer system of claim 12, wherein each door is coupled to the sanitizer container via a hinge.

15. The sanitizer system of claim 12, further comprising a pair of latches, each latch being used to secure one of the doors in the closed position, wherein each wipe storage compartment has an opening and wherein each latch includes a tab operatively coupled to the sanitizer dispenser and extending over the opening and adapted to secure the door in the closed position.

16. The sanitizer system of claim 12, wherein the doors are coupled to the sanitizer container via a living hinge.

17. The sanitizer system of claim 12, wherein the sanitizer container has an I-beam cross-section.

18. The sanitizer system of claim 12, wherein the sanitizer container comprises a web and flanges that form the wipe storage compartments.

19. The sanitizer system of claim 12, wherein the sanitizer dispenser comprises a clam-shell case that surrounds the sanitizer container and defines an opening from which the mechanical sprayer extends, wherein the clam-shell case is coupled via a snap-fit connection.

20. The sanitizer system of claim 12, wherein one or more of the doors has an access opening, and further comprising a gasket coupled to the door and defining the access opening.
